(19) United States
(12) Patent Application Publication
(10) Pub. No.: US 2012/0202831 A1
(43) Pub. Date: Aug. 9, 2012

(54) Pharmaceutical Formulation

(75) Inventors: Tsiala Benard, Sainte Genevieve Des Bois (FR); Jean-Pierre Burnouf, Paris (FR)

(73) Assignee: SANOFI, Paris (FR)

(21) Appl. No.: 13/361,309

(22) Filed: Jan. 30, 2012

Related U.S. Application Data
(63) Continuation of application No. PCT/FR2010/051611, filed on Jul. 29, 2010.

(30) Foreign Application Priority Data
Jul. 30, 2009 (FR) 0903742

(51) Int. Cl. A61K 31/519; A61P 35/00

(52) U.S. Cl. 514/264.11

(57) ABSTRACT

The invention relates to a pharmaceutical formulation including, as an active principle, the compound of formula (I)

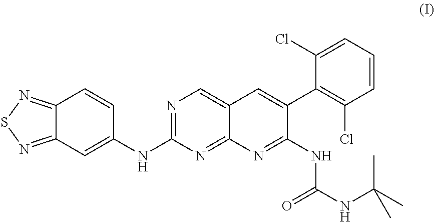

or a salt of said compound with a pharmaceutically acceptable acid.

### FIELD

The present application relates to pharmaceutical formulations.

### BACKGROUND

The administration of an anticancer agent intravenously is the preferred route of administration in oncology since it enables rapid diffusion of the agent in the bloodstream. It is sometimes the only form of administration when the agent does not exhibit sufficient bioavailability when it is administered via another route, such as orally.

The compound of formula (I) has a low solubility in various media (see Table I).

TABLE I

| medium | solubility [mg/g] |
|---|---|
| water | <0.006 |
| PEG 400 | 9.5 |
| propylene glycol | 1 |
| ethanol | 3.8 |
| tert-butanol | 1.2 |

However, it is necessary to be able to have a stable pharmaceutical formulation which can be manipulated and which makes it possible to administer a sufficient and effective amount of the compound of formula (I), of the order of a few hundred mg. It is also necessary for the pharmaceutical formulation to be chemically and physically stable, and likewise the perfusion solution obtained after dilution in an isotonic medium.

State of the art:WO 2007/003765 describes pyrido[2,3-d]pyrimidine derivatives of formula (A):

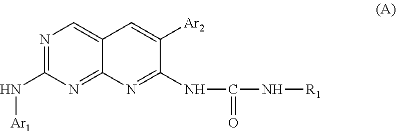

that can be administered in the form of a solution.

WO 08102075 describes the use of the compound of formula (I) in the treatment of leukaemias. The compound can be administered in the form of a solution. Said solution can be one of the following:
22% PEG 400/5% SOLUTOL® HS15/73% G5 for the iv treatment of mice bearing Kasumil tumours, G5 being a 5% solution of glucose in water;
21% LABRASOL®/5% SOLUTOL® HS15/74% 0.001 N HCl for the oral treatment of mice bearing Kasumi1 or KG1 tumours;
5% DMSO/10% TWEEN® 80/85% H₂O for the intraperitoneal treatment of mice bearing EOL-1 tumours.

The formulations described are therefore administered as they are to mice and are not intended to be diluted so as to form a perfusion solution.

### BRIEF DESCRIPTION OF THE INVENTION

The invention relates to a pharmaceutical formulation comprising the compound of formula (I) in the form of a base or in the form of a salt of an acid which is pharmaceutically acceptable, solubilized in a mixture of ethanol and of the surfactant Macrogol 15 hydroxystearate in a surfactant/ethanol ratio by weight ranging from 25/75 to 80/20, preferably from 73/27 to 77/23.

It is solubilized in a mixture of ethanol and of a surfactant comprising a mixture of the polyethoxylated monoester and diester of 12-hydroxystearic acid described hereinafter.

The surfactant comprises, by weight, from 35% to 55% of monoester and diester and from 30% to 40% of polyethylene glycol H(OCH₂CH₂)ₙ—OH. It comprises, by weight, as main components, from 35% to 55% of monoester and diester and from 30% to 40% of polyethylene glycol H(OCH₂CH₂)ₙ—OH, and also other compounds making up the rest to 100%. It comprises, by weight, from 10% to 20% of monoester, from 25% to 35% of diester and from 30% to 40% of polyethylene glycol H(OCH₂CH₂)—OH and also other compounds making up the rest to 100%.

The surfactant/ethanol ratio ranges from 73/27 to 77/23 and the concentration of compound of formula (I) ranges from 5 to 25 mg/ml.

The pharmaceutical formulation is intended to be diluted so as to form a perfusion solution.

The invention also relates to a method for preparing the pharmaceutical formulation, comprising the following steps:
heating the surfactant until it becomes liquid;
adding the ethanol;
cooling the surfactant/ethanol mixture to ambient temperature;
adding the compound of formula (I) to the cooled mixture;
sterilizing the final mixture, preferably by filtration.

The invention also relates to a perfusion solution comprising the compound of formula (I) in the form of a base or in the form of a salt of an acid which is pharmaceutically acceptable, obtained by diluting 1 volume of the pharmaceutical solution in 20 to 500 volumes of an isotonic solution. The compound of formula (I) at a concentration ranging from 0.01 to 1.2 mg/ml, the surfactant at a concentration ranging from 0.48 to 37 mg/ml and the ethanol at a concentration ranging from 0.35 to 35 mg/ml are diluted in the isotonic solution. The perfusion solution is intended to be administered to a human being.

The invention also relates to the method for preparing the perfusion solution, consisting in diluting 1 volume of the pharmaceutical solution in 20 to 500 volumes of the isotonic solution.

The invention also relates to a bottle containing the pharmaceutical solution and to a drip bag containing the perfusion solution.

The invention also relates to the use of a surfactant as defined above, for preparing a pharmaceutical formulation comprising the compound of formula (I) in the form of a base or in the form of a salt of an acid which is pharmaceutically acceptable, this formulation being intended to be diluted so as to form a perfusion solution.

### BRIEF DESCRIPTION OF THE DRAWINGS

FIG. 1: Shows a curve of change over time of the concentration of impurities for the SOLUTOL® HS15/ethanol formulation.

FIG. 2: Shows a curve of change over time of the concentration of impurities for the PS80/ethanol formulation.

FIG. 3: Shows a curve of change over time of the concentration of impurities for the PS80 formulation.

FIG. 4: Shows the technical information for SOLUTOL® HS15.

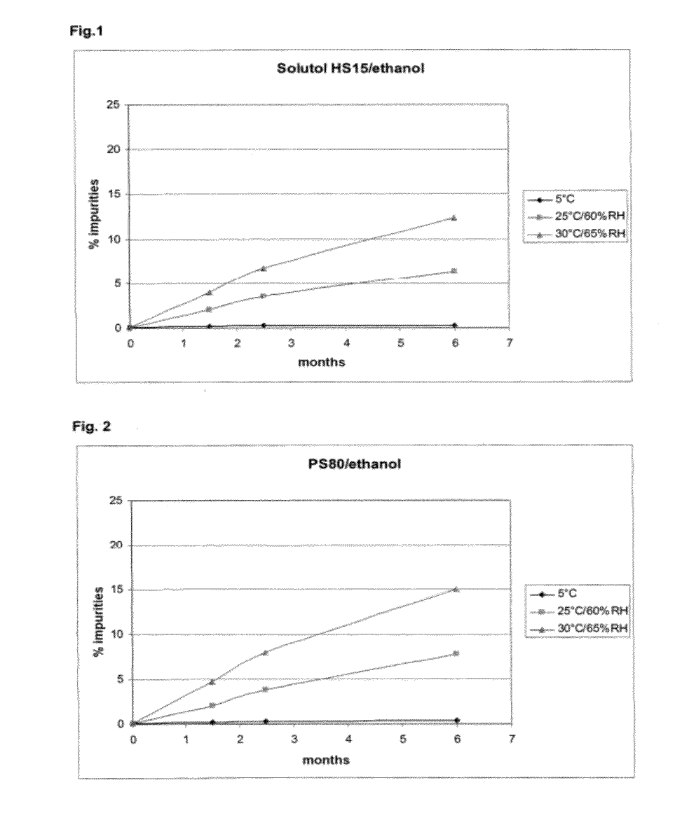

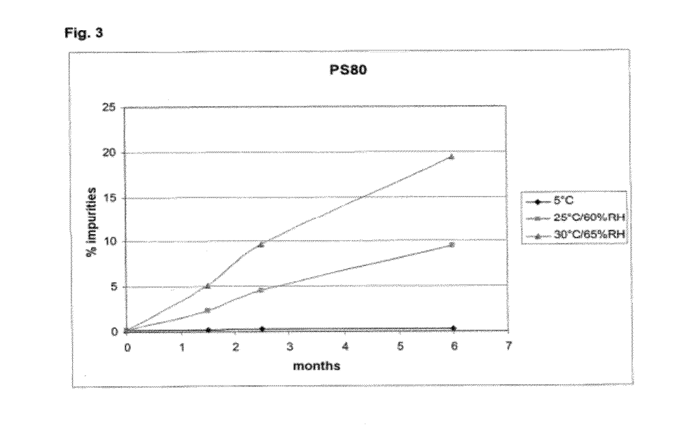

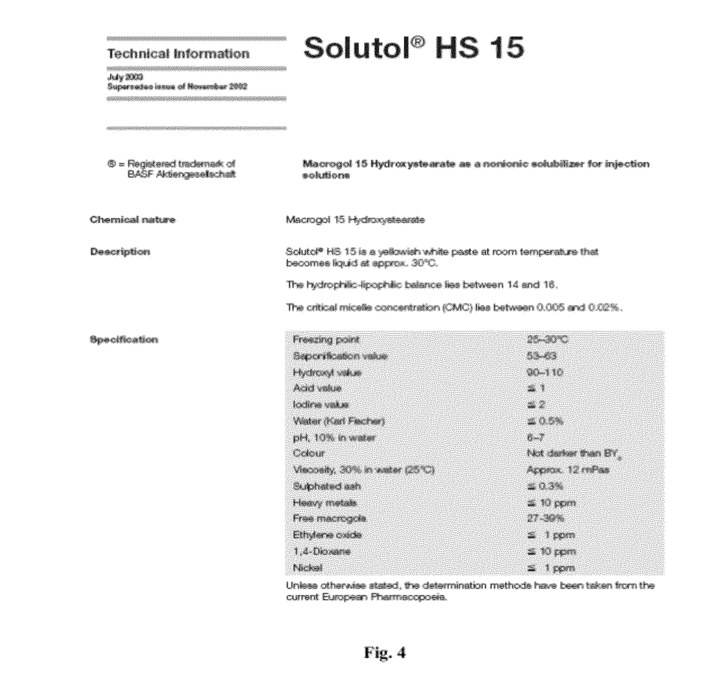

### DETAILED DESCRIPTION

The invention relates to a pharmaceutical formulation, comprising the compound of formula (I):

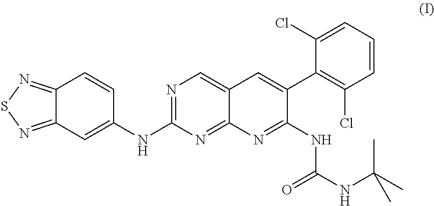

in the form of a base or in the form of a salt of an acid which is pharmaceutically acceptable, solubilized in a mixture of ethanol and of the surfactant Macrogol 15 hydroxystearate in a surfactant/ethanol ratio by weight ranging from 25/75 to 80/20, preferably from 73/27 to 77/23.

The pharmaceutical formulation comprises the compound of formula (I) solubilized in a mixture:
of ethanol and
of a surfactant comprising a mixture of the polyethoxylated monoester and diester of 12-hydroxystearic acid having the respective formulae:

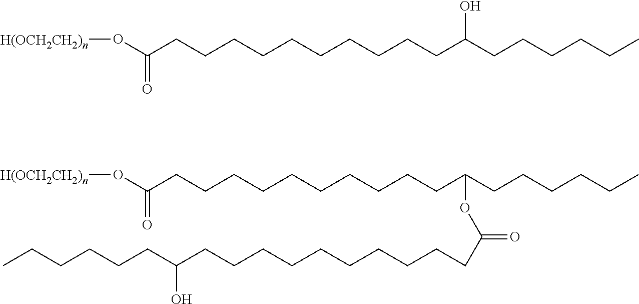

n being an integer ranging from 15 to 16, in a surfactant/ethanol ratio by weight ranging from 25/75 to 80/20, preferably from 73/27 to 77/23.

The compound of formula (I):

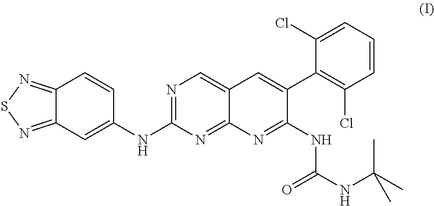

is an anticancer agent that can be used in the treatment of leukaemias, described in EP 1902054 B1. It may be in the form of a base (see formula (I)) or in the form of a salt of an acid which is pharmaceutically acceptable: in this respect, see “Remington's pharmaceutical sciences”, 17^{th} ed., Mack Publishing Company, Easton, Pa., 1985 and Berge et al., “Pharmaceutical salts” J. Pharm. Sci. 1977, 66, 1-19.

The non-ionic hydrophilic surfactant which is used is obtained by reacting, at about 110-165° C., 12-hydroxystearic acid and ethylene oxide in the presence of a basic catalyst such as K₂CO₃ according to the teaching of “Synthetic Detergents from Animal Fats. VIII. The Ethenoxylation of Fatty Acids and Alcohols” A. N. Wrigley J. Am. Oil. Chem. Soc. 1957, 34, 39-43 or of J. V. Karabinos J. Am. Oil Chem. Soc. 1954, 31, 20-23. The 12-hydroxystearic acid is derived from the hydrogenation of castor oil. See also EP 0017059.

The surfactant contains mainly a monoester of formula:

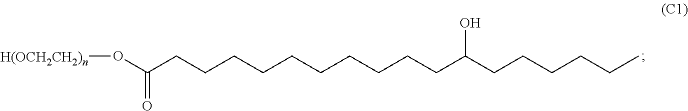

CAS No. 61909-81-7 and a diester of formula:

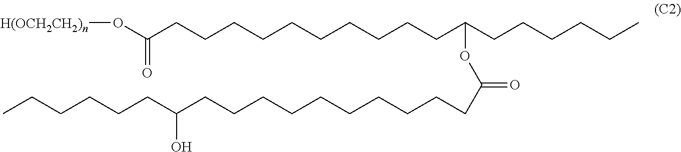

Moreover, the surfactant may also contain free polyethylene glycol (C3; H(OCH₂CH₂)₁₅₋₁₆—OH). The surfactant may thus comprise, by weight, from 30% to 40% of polyethylene glycol and from 60% to 70% of monoester and diester.

The surfactant may also comprise other compounds derived from the ethoxylation reaction, in particular those having the formulae:

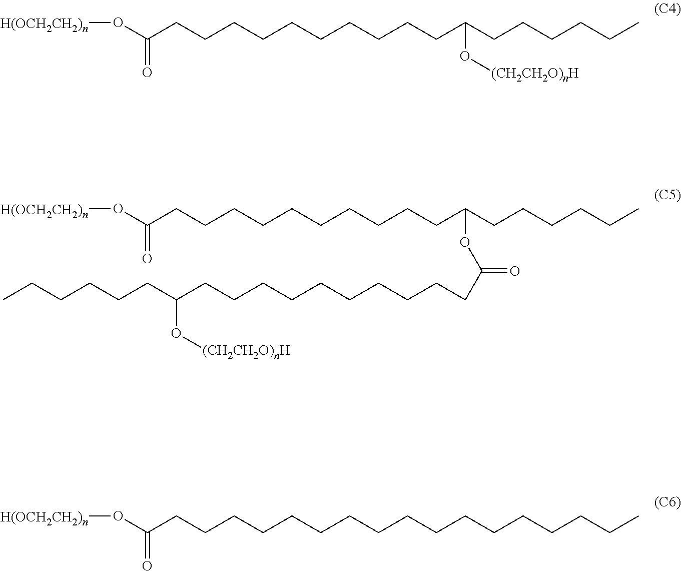

By way of examples, the compositions of two surfactants that can be used are given in Table II. The surfactant therefore comprises, by weight, as main components, from 35% to 55% of monoester and diester of 12-hydroxystearic acid and from 30% to 40% of polyethylene glycol H(OCH₂CH₂)ₙ—OH, and also other components making up the rest to 100%.

TABLE II

| Compounds | Surfactant | Surfactant | |
|---|---|---|---|
| [% weight] | example 1 | example 2 | Ranges |
| C1 (monoester) | 19 | 12 | 10-20 |
| C2 (diester) | 30 | 34 | 25-35 |
| C3 (PEG) | 35 | 35 | 30-40 |
| C4 | 9 | 6 | rest up to |
| C5 | 3 | 6 | 100%* |
| C6 | 3 | 2 | |
| other | 1 | 5 | |
| compounds | | | |

*for all the compounds C4-C6 + other compounds

An example of a surfactant that can be used is SOLUTOL® HS15 marketed by the company BASF, which is described in J. Pharm. Sci. 1998, 87(2), 200-208, Pharm. Res. 2004, 21(2), 201-230 (page 222), Int. J. Cancer 1995, 62, 436-442 and also in Cancer Res. 1991, 51, 897-902 and the technical information of which will be found in annexe 1. The European Pharmacopeia (PhEur 6.0) describes it as macrogol 15 hydroxystearate; it is described as a mixture of the monoester and diester of 12-hydroxystearic acid and of macrogol obtained by ethoxylation of 12-hydroxystearic acid. The number of moles of ethylene oxide having reacted with the acid is 15 (nominal value). It contains approximately 30% by weight of free macrogol. It is in the form of a whitish paste at ambient temperature which becomes liquid at approximately 30° C. The hydrophilic-lipophilic balance is approximately 14-16. The critical micelle concentration (CMC) lies between 0.005 and 0.12%. Other data: melting point: 25-30° C.; saponification value: 56-63; hydroxyl value: 90-110; iodine value: 2; viscosity at 30% by weight in water at 25° C.: approximately 12 mPa·s.

According to one embodiment of the invention, the pharmaceutical formulation may comprise at least one other additive customarily used in liquid pharmaceutical formulations. It may, for example, be an antioxidant, a preservative, a buffer, etc. According to another embodiment of the invention, the pharmaceutical formulation comprises only the surfactant, the ethanol and the compound of formula (I).

The pharmaceutical formulation can be prepared in the following way:

heating the surfactant until it becomes liquid;

The temperature at which the surfactant becomes liquid varies according to the surfactant and to the proportions of monoester and of diester and, where appropriate, of the free polyethylene glycol. The temperature is generally between 35 and 50° C. (limits included).

adding the ethanol;

The amount added is such that the surfactant/ethanol ratio is that given above.

cooling the surfactant/ethanol mixture to ambient temperature;

adding the compound of formula (I) to the cooled mixture;

sterilizing the final mixture. Filtration sterilization can advantageously be used: in this respect, see “Pharmaceutical process validation”, R. A. Nash, 3^{rd} edition, Marcel Dekker Inc, isbn=0824708385, page 119 or “Validation of pharmaceutical processes”, J. P. Agalloco, 3^{rd} edition, 2007, isbn=9780849370557, pages 151-152. Filtration sterilization does not degrade the compound of formula (I) which is heat-sensitive, unlike heating sterilization. For example, in the case of the formulation with a 75/25 ratio, it was possible to use filtration through a 0.22 μm filter.

The pharmaceutical formulation described above is a concentrate intended to be diluted so as to form a perfusion solution. It can be contained in a glass bottle. The perfusion solution is prepared extemporaneously by diluting the concentrate in an isotonic solution suitable for perfusion (for example, a solution containing glucose or a saline solution). The perfusion solution is generally prepared in the form of a perfusion drip by the hospital personnel just before administration. The perfusion solution is a supersaturated micellar solution of compound of formula (I) obtained by diluting 1 volume of concentrate in 20 to 500 volumes of isotonic solution. It can be used in the treatment of human cancers.

The function of the surfactant is to solubilize the compound of formula (I) in the formulation and to stabilize the perfusion solution (micellization). The solubility of the compound of formula (I) therefore increases as the surfactant/ethanol ratio increases. However, above the ratio 80/20, the viscosity of the formulation increases to the point of making it more difficult, or even impossible, to take a sample with a syringe.

The ethanol serves as a cosolvent and has the function of reducing the viscosity of the surfactant, thereby improving the manipulability thereof. Below a surfactant/ethanol ratio of 25/75, the amount of ethanol administered becomes considerable and the solubility of the compound of formula (I) becomes too low. The ethanol cannot be replaced with PEG 300 or 400 since the surfactant and the PEG 300/400 are not miscible at surfactant/PEG ratios ranging from 25/75 to 50/50 or alternatively the surfactant/PEG 300/400 mixture is solid at ambient temperature at ratios ranging from 60/40 to 75/25. Similarly, dilution of the compound of formula (I) in PEG alone does not make it possible to exceed a solubility of greater than 7 mg/ml, and the physical stability of the perfusion solution is not satisfactory (<24 h). Finally, the chemical stability studies have shown that the compound of formula (I) degrades less rapidly, especially under accelerated conditions at 25° C./60% RH (relative humidity) and 30° C./65% RH, in the formulation according to the invention than with a formulation based on PS80 or on PS80/ethanol.

The formulation according to the invention therefore offers the following advantages:

it is a homogeneous solution;

it makes it possible to achieve a sufficient solubility of compound of formula (I) to be able to administer, to a patient, an amount thereof of the order of a few hundred mg;

it can be manipulated and makes it possible in particular to take a sample with a syringe;

the compound of formula (I) does not degrade as much as with other surfactants (cf. Table IV);

the perfusion solution obtained using the formulation is physically stable for a period of at least 24 h at ambient temperature, i.e. it does not display any visible criterion of precipitation;

the formulation can be filter-sterilized.

The concentration of compound of formula (I) in the pharmaceutical formulation can range from 5 to 25 mg/ml. The solubility depends in fact on the surfactant/ethanol ratio. Examples of pharmaceutical formulations according to the invention are the following:

(A) surfactant/ethanol ratio from 73/27 to 77/23, for example of 75/25; compound of formula (I): 5-25 mg/ml;

(B) surfactant/ethanol ratio: 50/50; compound of formula (I): 5-10 mg/ml;

(C) surfactant/ethanol ratio: 25/75; compound of formula (I): 5 mg/ml.

The perfusion solution comprises the compound of formula (I) at a concentration ranging from 0.01 to 1.2 mg/ml, the surfactant at a concentration ranging from 0.48 to 37 mg/ml and the ethanol at a concentration ranging from 0.35 to 35 mg/ml, diluted in an isotonic solution. Preferably, it comprises the compound of formula (I) at a concentration ranging from 0.01 to 1.2 mg/ml, the surfactant at a concentration ranging from 1.4 to 35 mg/ml and the ethanol at a concentration ranging from 0.4 to 13 mg/ml, diluted in an isotonic solution.

### EXAMPLES

Preparation of a Formulation According to the Invention

In a glass reactor, the SOLUTOL® HS15 is melted at 40° C. for approximately 3 h, and then the heating is stopped and the vessel is made inert. The temperature of the vessel is brought down to 20° C. and, without waiting for the return to 20° C., the ethanol is added to the SOLUTOL® HS15. The mixture is then homogenized for 30 min. The compound of formula (I) (base form) is added and is dissolved in the SOLUTOL® HS15/ethanol mixture and the resulting mixture is left to stir for 3 h at ambient temperature. It is then filtered through a 0.22 μm PVDF filter and the solution is stored for 24 h. The solution is then subjected to a sterilizing filtration through a 0.22 μm PVDF filter.

Other Formulations

Several formulations of the compound of formula (I) are compared in order to determine the one which makes it possible to achieve the target solubility of 20 mg/g (Table III).

TABLE III

| Formulation based on | solubility [mg/g] | comments |
|---|---|---|
| PEG 400 | 9.5 | <target |
| propylene glycol | 1 | <target |
| ethanol | 3.8 | <target |
| tert-butanol | 1.2 | <target |
| mixed micelles (1/1 lecithin/Na | 0.004 | <target |
| taurocholate) 0.1M | | |
| 4% albumin | 0.013 | <target |
| 20% endolipid (emulsion) | 0.26 | <target |
| 20% medialipid (emulsion) | 0.13 | <target |
| 40% hydroxypropyl-beta-cyclodextrin | 0.087 | <target |
| Polysorbate 80 grade pH 6.0 (PS80_{pH 6}) | 29 | |
| Polysorbate 80 grade pH 3.5 (PS80_{pH 3.5}) | 32 | |
| PS80_{pH 6}/ethanol 75/25 (batch RSN2: | 27.5 | |
| purity = 99.7%) | | |
| PS80_{pH 6}/ethanol 75/25 (batch CER: | 30.3 | |
| purity = 95.6%) | | |
| PS80_{pH 6}/ethanol 50/50 | 21.2 | |
| SOLUTOL ® HS15/ethanol 50/50 | 26.9 | |
| SOLUTOL ® HS15/ethanol 75/25 (batch | 30.7 | |
| RSN2: purity = 99.7%) | | |
| SOLUTOL ® HS15/ethanol 75/25 (batch | 33.8 | |
| CER: purity = 95.6%) | | |
| SOLUTOL ® HS15/PEG400 20/80 | 14.4 | <target |

A study of chemical stability by measuring the impurity content by high performance liquid chromatography (HPLC) was carried out on the formulations making it possible to achieve the target solubility. To do this, the impurities present in the following three formulations were assayed over time:
SOLUTOL® HS15/ethanol 50/50
PS8O_{pH6}/ethanol 50/50
PS80

The results are given in Table IV.

TABLE IV

| | | SOLUTOL ® | | |
|---|---|---|---|---|
| conditions | months | HS15/ethanol | PS80/ethanol | PS80 |
| 5° C. | 0 | 0.13 | 0.13 | 0.15 |
| | 1.5 | 0.14 | 0.15 | 0.19 |
| | 2.5 | 0.26 | 0.29 | 0.25 |
| | 6 | 0.32 | 0.37 | 0.31 |
| 25° C./60% RH | 0 | 0.13 | 0.13 | 0.15 |
| | 1.5 | 2 | 2 | 2.3 |
| | 2.5 | 3.5 | 3.8 | 4.6 |
| | 6 | 6.4 | 7.8 | 9.5 |
| 30° C./65% RH | 0 | 0.13 | 0.13 | 0.15 |
| | 1.5 | 4 | 4.7 | 5.1 |
| | 2.5 | 6.7 | 8 | 9.7 |
| | 6 | 12.4 | 15 | 19.4 |

It is noted that the formulation combining SOLUTOL® HS15 and ethanol is the most stable of the three.

Physical and Chemical Stability of the Dilution in the Drip Bag

The SOLUTOL® HS15/ethanol 75/25 (weight/weight) concentrate is diluted extemporaneously in the drip bag. The physical and chemical stability of the dilution in the drip bag was studied. Various parameters were evaluated:
the dilution: 0.04 mg/mL and 1 mg/mL
the diluting medium (0.9% NaCl or 5% glucose)
the storage temperature (5° C. and 30° C.)
the storage time

Irrespective of the conditions tested, it was shown that the bags are chemically and physically stable for at least 72 h.

1. A pharmaceutical formulation comprising a compound of formula (I): 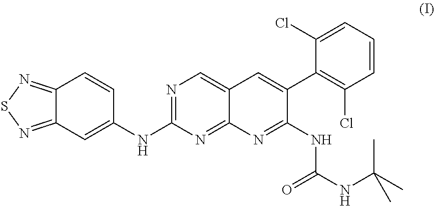
in the form of a base or in the form of a salt of an acid which is pharmaceutically acceptable,
wherein the compound is solubilized in a mixture of ethanol and a surfactant in a surfactant/ethanol ratio by weight ranging from 25/75 to 80/20, wherein the surfactant is macrogol 15 hydroxystearate.

2. The pharmaceutical formulation comprising a compound of formula (I): 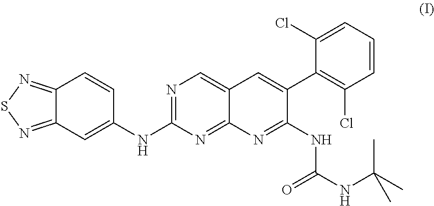
in the form of a base or in the form of a salt of an acid which is pharmaceutically acceptable,
wherein the compound is solubilized in a mixture of:
ethanol and
a surfactant comprising a mixture of a polyethoxylated monoester and diester of 12-hydroxystearic acid having the respective formulae: 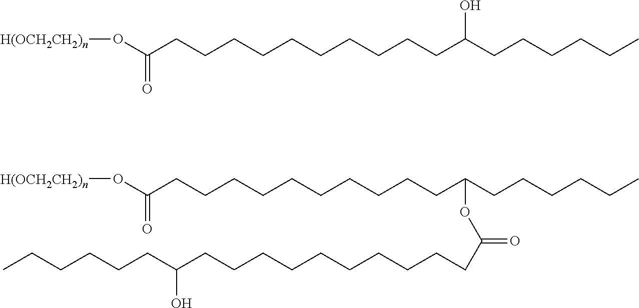
n being an integer ranging from 15 to 16,
in a surfactant/ethanol ratio by weight ranging from 25/75 to 80/20.

3. The pharmaceutical formulation according to claim 1, wherein the surfactant/ethanol ratio by weight ranges from 73/27 to 77/23.

4. The pharmaceutical formulation according to claim 2, wherein the surfactant comprises, by weight, from 35% to 55% of monoester and diester and from 30% to 40% of polyethylene glycol H(OCH₂CH₂)ₙ—OH.

5. The pharmaceutical formulation according to claim 4, wherein the surfactant comprises, by weight, from 35% to 55% of monoester and diester and from 30% to 40% of polyethylene glycol H(OCH₂CH₂)ₙ—OH.

6. The pharmaceutical formulation according to claim 1, wherein the surfactant comprises, by weight, from 10% to 20% of monoester, from 25% to 35% of diester and from 30% to 40% of polyethylene glycol H(OCH₂CH₂)ₙ—OH.

7. The pharmaceutical formulation according to claim 1, wherein the surfactant/ethanol ratio ranges from 73/27 to 77/23 and the concentration of compound of formula (I) ranges from 5 to 25 mg/ml.

8. The pharmaceutical formulation according to one of claim 1, wherein the formulation is suitable to be diluted so as to form a perfusion solution.

9. A method for preparing a pharmaceutical formulation comprising a compound of formula (I): 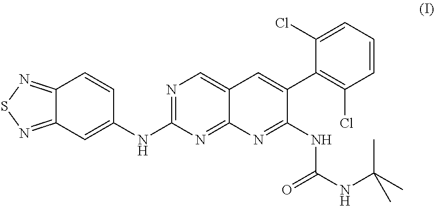
in the form of a base or in the form of a salt of an acid which is pharmaceutically acceptable, comprising:
heating the surfactant until it becomes liquid;
adding the ethanol;
cooling the surfactant/ethanol mixture to ambient temperature;
adding the compound of formula (I) to the cooled mixture;
sterilizing the final mixture.

10. A method according to claim 9, wherein the mixture is sterilized by filtration.

11. A perfusion solution comprising a compound of formula (I): 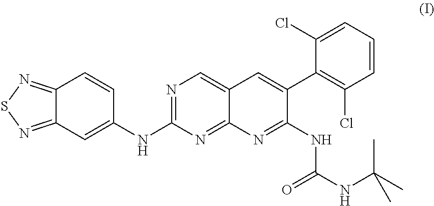
in the form of a base or in the form of a salt of an acid which is pharmaceutically acceptable,
obtained by diluting 1 volume of the pharmaceutical formulation according to claim 1 in 20 to 500 volumes of an isotonic solution.

12. A perfusion solution comprising the compound of formula (I): 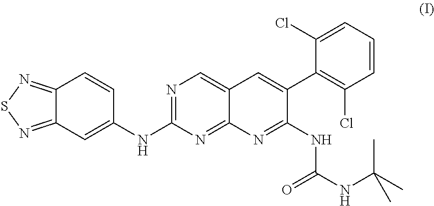
in the form of a base or in the form of a salt of an acid which is pharmaceutically acceptable, at a concentration ranging from 0.01 to 1.2 mg/ml, a surfactant
at a concentration ranging from 0.48 to 37 mg/ml, ethanol at a concentration ranging from 0.35 to 35 mg/ml, and isotonic solution, wherein the surfactant is macrogol 15 hydroxystearate.

13. The perfusion solution according to claim 11, suitable to be administered to a human being.

14. A perfusion solution comprising the compound of formula (I): 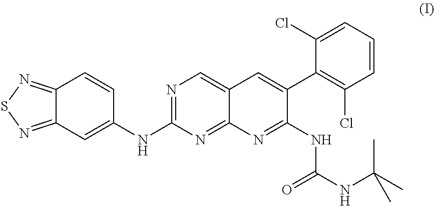
in the form of a base or in the form of a salt of an acid which is pharmaceutically acceptable, at a concentration ranging from 0.01 to 1.2 mg/ml, a surfactant at a concentration ranging from 0.48 to 37 mg/ml, ethanol at a concentration ranging from 0.35 to 35 mg/ml, and an isotonic solution, wherein the surfactant comprises a mixture of a polyethoxylated monoester and diester of 12-hydroxystearic acid having the respective formulae: 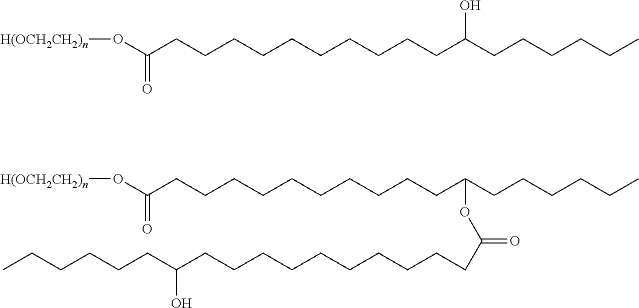
n being an integer ranging from 15 to 16, in a surfactant/ethanol ratio by weight ranging from 25/75 to 80/20.

15. A method for preparing a perfusion solution, comprising diluting 1 volume of a pharmaceutical formulation 20to 500 volumes of an isotonic solution, wherein the pharmaceutical formulation comprises a compound of formula (I): 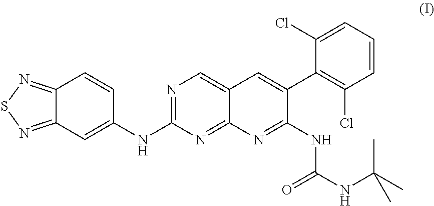
in the form of a base or in the form of a salt of an acid which is pharmaceutically acceptable, solubilized in a mixture of ethanol and a surfactant in a surfactant/ethanol ratio by weight ranging from 25/75 to 80/20, wherein the surfactant is macrogol 15 hydroxystearate.

16. A method for preparing a perfusion solution comprising diluting 1 volume of a pharmaceutical formulation in 20 to 500 volumes of an isotonic solution, wherein the pharmaceutical formulation comprises a compound of formula (I): 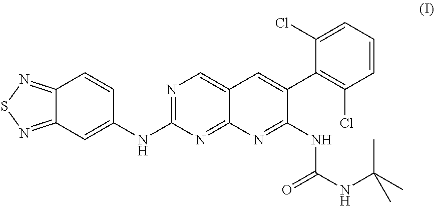
in the form of a base or in the form of a salt of an acid which is pharmaceutically acceptable, solubilized in a mixture of ethanol and a surfactant in a surfactant/ethanol ratio by weight ranging from 25/75 to 80/20, wherein the surfactant comprises a mixture of a polyethoxylated monoester and diester of 12-hydroxystearic acid having the respective formulae: 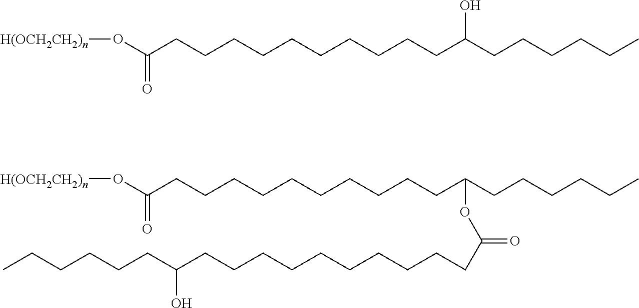
n being an integer ranging from 15 to 16.

17. A bottle containing a pharmaceutical formulation, wherein the pharmaceutical formulation comprises a compound of formula (I): 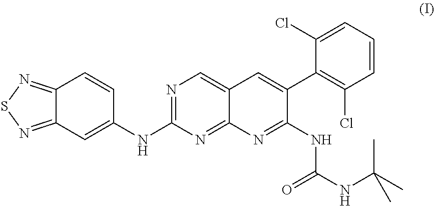
in the form of a base or in the form of a salt of an acid which is pharmaceutically acceptable, solubilized in a mixture of ethanol and a surfactant in a surfactant/ethanol ratio by weight ranging from 25/75 to 80/20, wherein the surfactant is macrogol 15 hydroxystearate

18. A drip bag containing a perfusion solution, wherein the perfusion solution comprises the compound of formula (I): 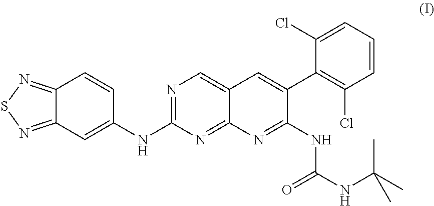
in the form of a base or in the form of a salt of an acid which is pharmaceutically acceptable, at a concentration ranging from 0.01 to 1.2 mg/ml, a surfactant
at a concentration ranging from 0.48 to 37 mg/ml, ethanol at a concentration ranging from 0.35 to 35 mg/ml, and isotonic solution, wherein the surfactant is macrogol 15 hydroxystearate.
